(19) United States
(12) Patent Application Publication
(10) Pub. No.: US 2021/0388018 A1
(43) Pub. Date: Dec. 16, 2021

(54) METHYL AND ETHYL NICOTINATE-RIBOSIDE-5-PHOSPHATES, PREPARATION THEREOF AND METHODS OF USE THEREOF

(71) Applicant: Cornell University, Ithaca, NY (US)

(72) Inventor: Anthony A. Sauve, New Rochelle, NY (US)

(73) Assignee: Cornell University, Ithaca, NY (US)

(21) Appl. No.: 17/281,632

(22) PCT Filed: Oct. 1, 2019
(86) PCT No.: PCT/US2019/054073

§ 371 Date: Mar. 31, 2021

Related U.S. Application Data
(60) Provisional application No. 62/739,567, filed on Oct. 1, 2018.

(51) Int. Cl. C07H 19/048; A23L 33/13; C07H 1/02

(52) U.S. Cl. CPC C07H 19/048; C07H 1/02; A23L 33/13

(57) ABSTRACT

The invention provides a compound of formula (I): (I) wherein R is methyl or ethyl. The invention also provides a process for the preparation of the compound. The invention further provides a method for increasing cell NAD⁺ production or improving mitochondrial densities in a cell, wherein the method comprises administering to the cell a compound or salt of the invention.

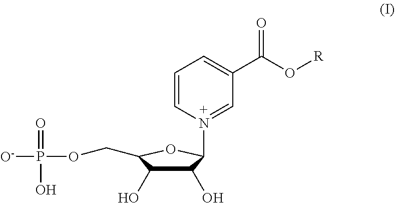

### CROSS-REFERENCE TO A RELATED APPLICATION

This patent application claims the benefit of U.S. Provisional Patent Application No. 62/739,567, filed October 1, 2018, which is incorporated by reference in its entirety for all purposes.

### BACKGROUND OF THE INVENTION

Nicotinamide adenine dinucleotide (NAD) is an important co-enzyme and substrate in several biological pathways and biochemical reactions including ADP-ribosylation and protein deacetylation and as an essential redox co-factor for many enzymes. NAD participation in metabolism makes it an important metabolite in several biological processes, such as aging, apoptosis, DNA repair, transcriptional regulation, and immune response.

NAD can be synthesized from different precursors containing pyridine moieties in several salvage pathways (nicotinamide (Nam), nicotinic acid (NA), and nicotinamide riboside (NR)) and in the de novo pathway from tryptophan. Many non-mammalian organisms use nicotinic acid (a form of vitamin B₃) as a major NAD precursor. Mammals predominantly use nicotinamide (another form of vitamin B₃) for NAD biosynthesis. In bacteria and yeast, Nam is converted to NA by the enzyme nicotinamidase, but this enzyme is not encoded in mammal genomes.

Cellular NAD consumption is high, and variation in the cellular levels of NAD plays an important role in health and diseases like cancer, diabetes, neurodegenerative diseases, and autoimmune disorders. Constant recycling of NAD is crucial to sustain the activities of cellular enzymes. In mammals, particularly in humans, the main source of cellular NAD is from salvage pathways, which require the uptake or metabolism of NAD precursors (i.e., NAM, NA, nicotinate mononucleotide (NaMN), nicotinamide mononucleotide (NMN), and nicotinamide riboside (NR)) from the diet or via intracellular reuse after metabolism. Efficient chemical syntheses of these precursors are in high demand. U.S. Pat. No. 8,106,184 describes ester derivatives of nicotinic acid riboside and their ability to increase intracellular NAD in HeLa cells. Thus, stereoisomerically pure 5′-phosphates of nicotinate ester ribosides may be effective to treat a disease or disorder that would benefit from increased NAD levels, including insulin resistance, obesity, diabetes, and metabolic syndrome. An efficient synthetic route to 5′-phosphates of nicotinate ester ribosides is needed to obtain and evaluate such compounds for their utility as precursors to NAD.

A commonly practiced method for the synthesis of 5′-ribotides from the corresponding ribosides employs a protection and deprotection strategy, which involves protection of secondary alcohols followed by phosphorylation of 5′-hydroxy group and then deprotection of the secondary alcohols. This strategy is inefficient in terms of time, cost, and particularly yields.

Thus, there remains in the art a need for an efficient synthesis of 5′-phosphates of nicotinate ester ribosides.

### BRIEF SUMMARY OF THE INVENTION

The invention provides a compound of formula (I):

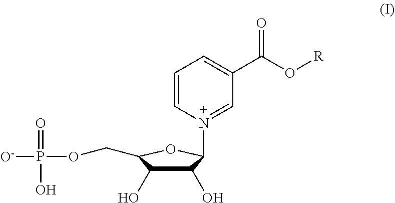

wherein R is methyl or ethyl, or a salt thereof.

The invention also provides a method for increasing cell NAD⁺ production comprising administering to a cell a compound of formula (I):

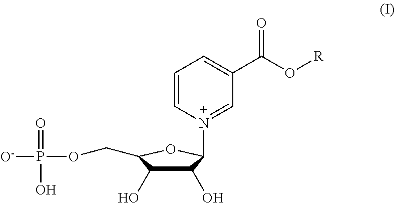

wherein R is methyl or ethyl, or a salt thereof

The invention further provides a method of improving mitochondrial densities in a cell, wherein the method comprises administering to the cell a compound of formula (I):

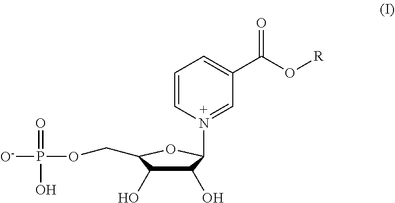

wherein R is methyl or ethyl, or a salt thereof

The invention additionally provides a process for the preparation of a compound of formula (I):

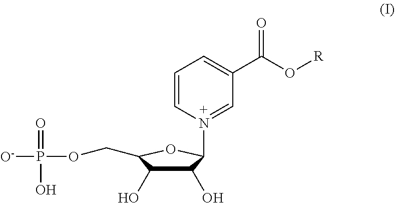

wherein R is methyl or ethyl, or a salt thereof, wherein the process comprises the steps of:

(i) reacting a nicotinate ester (IV):

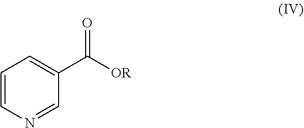

with 1,2,3,4-tetra-O-acetyl-D-ribofuranose to provide a compound of formula (V):

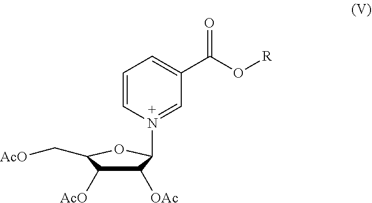

(ii) reacting the compound of formula (V) with a base to form the compound of formula (III):

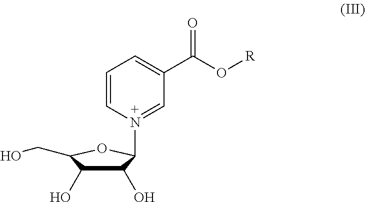

and
(iii) reacting the compound of formula (III) with a mixture of POCl₃ and PO(OR⁵)₃, wherein R⁵ is C₁-C₆ alkyl, followed by treatment with water to form the compound of formula (I).

The invention also provides a process for the preparation of a compound of formula (I):

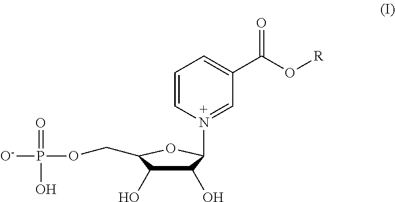

wherein R is methyl, or a salt thereof, wherein the process comprises the steps of:

(i) reacting a nicotinate ester (IV):

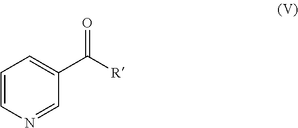

with 1,2,3,4-tetra-O-acetyl-D-ribofuranose to provide a compound of formula (VI):

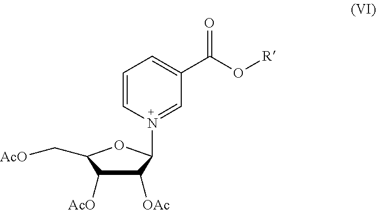

(ii) reacting the compound of formula (VI) with a base to form the compound of formula (VII):

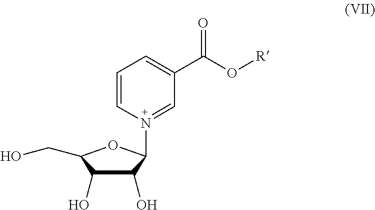

(iii) reacting the compound of formula (VII) with a mixture of POCl₃ and PO(OR⁵)_{3,} wherein R⁵ is C₁-C₆ alkyl, followed by treatment with water to form the compound of formula (VIII):

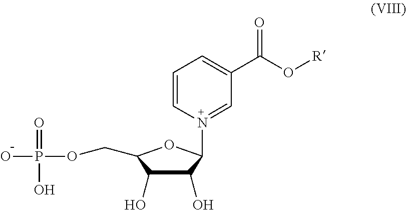

and

(iv) reacting the compound of formula (VIII) with sodium methoxide to form the compound of formula (I).

### BRIEF DESCRIPTION OF THE SEVERAL VIEWS OF THE DRAWINGS

FIG. 1 is a bar graph showing the effect on intracellular levels of NAD in HEK293 and Neuro2a cells on treatment with NaMN and NR.

FIGS. 2A-2D show the effects of 4 hour intraperitoneal injections in mice of O-ethyl nicotinamide riboside and O-methyl nicotinamide riboside in tissue NAD concentrations in liver (FIG. 2A), blood (FIG. 2B), skeletal muscle (FIG. 2C), and kidney (FIG. 2D).

### DETAILED DESCRIPTION OF THE INVENTION

In an embodiment, the invention provides a compound of formula (I):

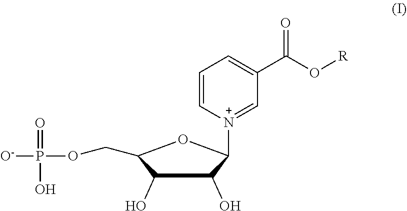

wherein R is methyl or ethyl, or a salt thereof.

In a particular embodiment, the compound is:

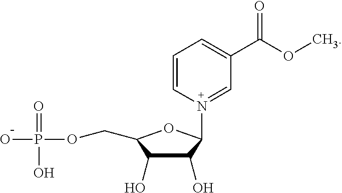

In another particular embodiment, the compound is:

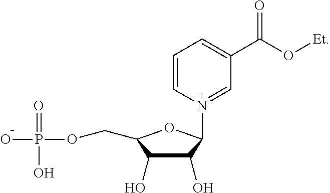

The phrase “salt” or “pharmaceutically acceptable salt” is intended to include nontoxic salts, which can be synthesized from the parent compound, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, a nonaqueous medium, such as ether, ethyl acetate, ethanol, isopropanol, or acetonitrile, is preferred. Lists of suitable salts are found in Remington's Pharmaceutical Sciences, 18th ed., Mack Publishing Company, Easton, Pa., 1990, p. 1445, and Journal of Pharmaceutical Science, 66: 2-19 (1977). For example, a suitable salt can be a salt of an alkali metal (e.g., sodium or potassium), alkaline earth metal (e.g., calcium), or salt of ammonium or alkylammonium, for example, monoalkylammonium, dialkylammonium, trialkylammonium, or tetraalkylammonium.

Examples of pharmaceutically acceptable salts for use in the inventive pharmaceutical composition include those derived from mineral acids, such as hydrochloric, hydrobromic, phosphoric, metaphosphoric, nitric and sulphuric acids, and organic acids, such as tartaric, acetic, citric, malic, lactic, fumaric, benzoic, glycolic, gluconic, succinic, maleic and arylsulfonic acids, for example, methanesulfonic, trifluoromethanesulfonic, benzenesulfonic, and p-toluenesulfonic acids.

The invention further provides a composition, preferably a pharmaceutical composition, comprising a compound as described above in any of the embodiments and a suitable carrier, preferably a pharmaceutically acceptable carrier. The invention provides a pharmaceutical composition comprising a pharmaceutically acceptable carrier and an effective amount, e.g., a therapeutically effective amount, including a prophylactically effective amount, of one or more of the aforesaid compounds, or salts thereof, of the invention.

The pharmaceutically acceptable carrier can be any of those conventionally used and is limited only by chemico-physical considerations, such as solubility and lack of reactivity with the compound, and by the route of administration. In addition to the following described pharmaceutical compositions, the compounds of the invention can be formulated as inclusion complexes, such as cyclodextrin inclusion complexes, or liposomes.

The pharmaceutically acceptable carriers described herein, for example, vehicles, adjuvants, excipients, or diluents, are well known to those who are skilled in the art and are readily available to the public. The pharmaceutically acceptable carrier preferably is chemically inert to the active compounds and has no detrimental side effects or toxicity under the conditions of use.

The choice of carrier will be determined in part by the particular active agent, as well as by the particular method used to administer the composition. Accordingly, there is a wide variety of suitable formulations of the pharmaceutical composition of the invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the invention, alone or in combination with other suitable components, can be made into aerosol formulations to be administered via inhalation. These aerosol formulations can be placed into pressurized acceptable propellants, such as dichlorodifluoromethane, propane, nitrogen, and the like. The compounds of the invention also may be formulated as pharmaceuticals for non-pressured preparations, such as in a nebulizer or an atomizer

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compounds of the invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the invention may be made into injectable formulations. The requirements for effective pharmaceutical carriers for injectable compositions are well known. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

Additionally, the compounds of the invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate.

The invention also provides a nutraceutical composition comprising a compound of the invention. The term nutraceutical as used herein denotes the usefulness in both the nutritional and pharmaceutical field of application. The nutraceutical compositions according to the invention may be in any form that is suitable for administrating to the animal body including the human body, especially in any form that is conventional for oral administration, e.g. in solid form such as (additives/supplements for) food or feed, food or feed premix, tablets, pills, granules, dragees, capsules, and effervescent formulations such as powders and tablets, or in liquid form such as solutions, emulsions or suspensions as e.g. beverages, pastes and oily suspensions. Controlled (delayed) release formulations incorporating the compounds according to the invention also form part of the invention. Furthermore, a multi-vitamin and mineral supplement may be added to the nutraceutical compositions of the invention to obtain an adequate amount of an essential nutrient, which is missing in some diets. The multi-vitamin and mineral supplement may also be useful for disease prevention and protection against nutritional losses and deficiencies due to lifestyle patterns.

In an embodiment, the invention provides a method for increasing cell NAD⁺ production comprising administering a compound of the invention or a salt thereof to a cell. In certain embodiments, the cell is in a mammal having a lipid disorder, a metabolic dysfunction, a cardiovascular disease, CNS or PNS trauma, a neurodegenerative disease or condition, or hearing loss, or is in a mammal that has been exposed to a toxic agent. In certain embodiments, the cell is in a mammal at risk for hearing loss. In certain other embodiments, the cell is in a mammal, and the compound is administered in an amount effective for promoting the function of the metabolic system, promoting muscle function or recovery, promoting the function of the auditory system, or promoting cognitive function

In another embodiment, the invention provides a method of improving mitochondrial density in a cell, wherein the method comprises administering to the cell a compound of the invention or a salt thereof. In certain embodiments, the cell is in a mammal having a lipid disorder, a metabolic dysfunction, a cardiovascular disease, CNS or PNS trauma, a neurodegenerative disease or condition, hearing loss, or is in a mammal that has been exposed to a toxic agent. In certain embodiments, the cell is in a mammal at risk for hearing loss. In certain other embodiments, the cell is in a mammal, and the compound is administered in an amount effective for promoting the function of the metabolic system, promoting muscle function or recovery, promoting the function of the auditory system, or promoting cognitive function.

Exemplars of the compounds of the invention exhibit a surprising and unexpected effect on mammalian tissues vis-a-vis NAD⁺ increases. This effect occurs at doses of 100-1000 mg/kg where other compounds at any concentration are not efficacious to achieve the effect. Because of esterification, the compounds are also more lipophilic than their respective unesterified relatives, which may provide for increased absorption and blood-brain-barrier (BBB) penetration characteristics. Key features of the compounds are potency, ease of access, improved biological efficacy in enhancing NAD⁺, and opportunities for improved drug behavior from enhanced lipophilicity.

In some embodiments, the invention provides a method for increasing mammalian cell NAD⁺ production comprising administering a compound of the invention or a pharmaceutically acceptable salt thereof to a cell. Nicotinamide adenine dinucleotide (NAD or NAD⁺) is important as a co-enzyme for different enzymes. Recent studies depicted that, being the co-substrate of SIR2 (silent information regulator 2), NAD⁺ has a role in regulating multiple biological processes, such as p53 regulated apoptosis, fat storage, stress resistance, and gene silencing. Without limiting the potential uses of the compositions described herein by any single theory, there are various pathways through which nicotinamide riboside (NR), dihydronicotinamide riboside (NRH), nicotinic acid riboside (NAR), and dihydronicotinic acid riboside (NARH or NaR—H) as well as nicotinic acid mononucleotide (NaMN) and their derivatives are currently thought to be metabolized. NR is a known as NAD⁺ precursors for both human and yeast. NR is able to enter a salvage pathway that leads to biological synthesis of NAD⁺ under the action of the enzyme nicotinamide riboside kinase (Nrk). NR can be converted to nicotinamide mononucleotide (NMN) whereas nicotinic acid riboside (NaR) is converted to nicotinic acid mononucleotide (NAMN) by respective phosphorylations mediated by nicotinamide riboside kinases (Nrk). The mononucleotides are then converted to corresponding dinucleotides NAD⁺ and nicotinic acid adenine dinucleotide (NaAD) by the enzyme nicotinamide mononucleotide adenylytransferase (Nmnat). Alternatively, NR and NAR can enter NAD metabolism by means of other metabolic paths, which include action from enzymes that separate the nicotinamide or nicotinic acid moiety from the sugar. Such a path includes the action of phosphorylases that have been shown to degrade NR and NaR in cells to form nicotinamide and nicotinic acid respectively, and ribose-1-phosphate. Both nicotinamide and nicotinic acid are competent to enter NAD⁺ metabolism and be converted to NAD⁺ by the action of the enzymes nicotinamide pyrophosphoribosyltransferase and nicotinic acid phosphoribosyltransferase respectively, to form NMN and NaMN respectively. Downstream of NAD are other enzymes which mediate NAD effects. For example, sirtuins are class III histone deacetylases (HDACs) and also are ADP-ribosyl transferases. Sirtuins deacetylate lysine residues in a novel chemical reaction that consumes nicotinamide adenine dinucleotide (NAD⁺) releasing nicotinamide, O-acetyl-ADPribose (AADPR), and the deacetylated substrate. By these activities, and by altering intracellular NAD⁺ levels, one can improve the health of a cell, but introduction of compounds that enter NAD⁺ metabolic pathways can also prove toxic to cells. In some embodiments, the invention relates to the use of compounds disclosed herein to manipulate NAD⁺ levels, to modulate the activity of sirtuins and other ADP-ribosyl transferases, and to modulate inosine 5′-monophosphate dehydrogenase. These embodiments are used to destroy or weaken the defenses of cancer cells, or to promote survival of neurons, myocytes, or stem cells via addition to growth media.

Nicotinic acid is an effective agent in controlling low-density lipoprotein cholesterol, increasing high-density lipoprotein cholesterol, and reducing triglyceride and lipoprotein (a) levels in humans. Though nicotinic acid treatment affects all of the key lipids in the desirable direction and has been shown to reduce mortality in target populations, its use is limited because of a side effect of heat and redness termed flushing. Further, nicotinamide is neuroprotective in model systems, presumably due to multiple mechanisms including increasing mitochondrial NAD⁺ levels.

In addition, NR and derivatives thereof have proved useful in model systems and in clinical trials in humans for a variety of uses, including promoting healthy aging, supporting and promoting healthy metabolic function, supporting and promoting cognitive function, neuroprotection in CNS and PNS trauma including stroke, and in neurogenerative diseases and conditions including essential tremor, Parkinson disease, Alzheimer disease, Huntington disease, ataxia, catatonia, epilepsy, neuroleptic malignant syndrome, dystonia, neuroacanthocytosis, Pelizaeus-Merzbacher, progressive supranuclear palsy, Striatonigral degeneration, Tardive dyskinesias, lysosomal storage disorders, including lipid storage disorders (including Gaucher's and Niemann-Pick diseases), gangliosidosis (including Tay-Sachs disease), leukodystrophies, mucopolysaccharidoses, glycoprotein storage disorders, and mucolipidoses. NR and derivatives thereof have been found useful to prevent hearing loss due to aging or exposure to loud sounds. NR and derivatives thereof can protect cells from damage to exposure to toxins, including damage to myocytes caused by statins. NR and derivatives thereof can slow or prevent the death of islet cells that produce insulin. NR and derivatives thereof have been found to increase the number of, and improve the function of, mitochondria.

NaMN derivatives may be bioavailable and are ultimately convertible by metabolism to nicotinic acid or nicotinic acid riboside (NAR), nicotinic acid mononucleotide (NaMN), Nicotinic acid adenine dinucleotide (NaAD) and ultimately to NAD+, thereby providing the benefits of the compounds as discussed above. Accordingly, one embodiment of the invention relates to the use of compositions comprising compounds disclosed herein that work through the nicotinamide riboside kinase pathway or other pathways of NAD⁺ biosynthesis which have nutritional and/or therapeutic value in improving poor plasma lipid profiles in lipid disorders, (e.g., dyslipidemia, hypercholesterolaemia or hyperlipidemia), metabolic dysfunction in type I and II diabetes, cardiovascular disease, and other physical problems associated with obesity, protecting islet cells to treat or prevent development of diabetes, neuroprotection to treat trauma and neurodegenerative diseases and conditions, protecting muscle cells from toxicity and damage from workouts or trauma, promoting the function of the auditory system, treating or preventing hearing loss, and dietary supplement and food ingredient uses for promoting metabolic function, muscle function and healing/recovery, cognitive function, and mitochondrial function.

In some embodiments, the invention relates to the use of compounds disclosed herein as agonists and antagonists of enzymes in the pathway of NAD⁺ biosynthesis. In further embodiments, the NaMN derivatives disclosed herein are agonists, i.e., stimulate activities normally stimulated by naturally occurring substances, of one or more sirtuins, preferably SIRT1 in humans or Sir2p in yeast. In further embodiments, the NaMN derivatives are antagonists of one or more of the sirtuins.

In some embodiments, the invention provides a method of improving metabolic function, including increased mitochondrial densities, insulin sensitivity, or exercise endurance in a mammal, wherein the method comprises administering to the mammal a compound of the invention or a pharmaceutically acceptable salt, or salt acceptable for dietary supplements or food ingredients, thereof. Under calorie restriction, cellular energy depletion causes rising AMP levels, and an increase in the NAD⁺ level as compared to the reduced level (NADH) results in activation of AMPK. AMPK activation leads to PGC-1alpha activation which leads to mitochondrial biosynthesis (Lopez-Lluch, et al., Experimental Gerontology, 43 (9): 813-819 (2009) [doi:10.1016/j.exger.2008.06.0141]). Increasing mitochondrial biosynthesis will lead to increased mitochondrial density in the muscle cells. Increased mitochondrial density will increase athletic performance in terms of muscle strength and endurance.

In some embodiments, the invention provides a method of treating or preventing a disease or condition in a mammal in need thereof, wherein the method comprises administering to the mammal a compound of the invention or a pharmaceutically acceptable salt thereof, wherein the disease or condition is CNS or PNS trauma, or a neurodegenerative disease or condition.

NAD⁺ levels decrease in injured, diseased, or degenerating neural cells and preventing this NAD⁺ decline efficiently protects neural cells from cell death. Araki & Milbrandt, Science, 305(5686): 1010-1013 (2004), and Wang et al., “A local mechanism mediates NAD-dependent protection of axon degeneration,” J. Cell Biol., 170(3): 349-55(2005), hereby incorporated by reference. As a number of inventive compounds disclosed herein are capable of increasing intracellular levels of NAD⁺, these compounds are useful as a therapeutic or nutritional supplement in managing injuries, diseases, and disorders effecting the central nervous system and the peripheral nervous system, including but not limited to trauma or injury to neural cells, diseases or conditions that harm neural cells, and neurodegenerative diseases or syndromes. Some neurodegenerative diseases, neurodegenerative syndromes, diseases, conditions that harm neural cells, and injury to neural cells are described above. The inventive compounds disclosed herein preferably are capable of passing the blood-brain-barrier (BBB).

In some embodiments, the invention provides a method of protecting a mammal from neurotrauma, wherein the method comprises administering to the mammal a compound of the invention or a pharmaceutically acceptable salt thereof. In certain of these embodiments, the neurotrauma results from blast injury or noise. In these embodiments, the agent increases intracellular NAD⁺ in one or more cells selected from the group consisting of spiral ganglia nerve cells, hair cells, supporting cells, and Schwann cells.

In certain embodiments, the agent suppresses oxidative damage in the cell. In certain embodiments, the compound activates SIRT3. Endogenous SIRT3 is a soluble protein located in the mitochondrial matrix. Overexpression of SIRT3 in cultured cells increases respiration and decreases the production of reactive oxygen species. Without wishing to be bound by any particular theory, it is believed that activation of SIRT3 is implicated in suppression of oxidative damage in the aforesaid cells.

In certain embodiments, the treating of the mammal with the compound results in prevention of hearing loss. In other embodiments, the treating of the mammal with the agent results in the mitigation of hearing loss. The treating can be performed after exposure to the mammal to circumstances leading to hearing loss, such as exposure to noise, or can be performed prior to exposure of the mammal to the circumstances. The relationship of NAD⁺ levels and protection from neurotrauma is disclosed in WO 2014/014828 A1, the contents of which are incorporated herein by reference. In certain embodiments, the compound supports the healthy structure or function of the auditory system in a mammal in need thereof. Treating of the mammal with an effective amount of the compound, for example, in a dietary supplement or in a food ingredient composition, augments intracellular NAD⁺ biosynthesis, wherein intracellular NAD⁺ increases in spiral ganglia nerve cells, hair cells, supporting cells, Schwann cells, or a combination thereof. In some embodiments, the agent maintains axonal NAD+ levels following axonal injuries caused by acoustic trauma.

Statins, more mechanistically known as 3-hydroxy-3-methyglutaryl coenzyme A reductase inhibitors (or HMG-CoA inhibitors), are some of the world's most widely prescribed drugs. While statins are well tolerated at therapeutic doses, at higher doses and often in combination with other hypolipidaemic agents some potentially severe adverse effects have arisen. Most notably, cerivastatin (Baycol) was removed from the market in 2000 after 31 deaths in the United States from drug-associated rhabdomyolysis (breakdown of muscle fibers resulting in the release of muscle fiber contents into the circulation; some of these are toxic to the kidney) and associated acute renal failure in patients taking cerivastatin. Statins are also known to have severe interactions with fibric acid derivatives, especially with gemfibrozil. Of the 31 people who died taking cerivastatin, 12 were also taking gemfibrozil.

The most serious adverse effects of statins appear to occur in liver and muscle cells, although it could be predicted that because of their lipophilicity, cerebral effects might also be seen in some patients.

The exact mechanism of statin toxicities is unknown. The fact that toxicities are dose-dependent makes plausible the hypothesis that toxicities result from exaggeration of the drug's intended effect. In other words, cells die from lack of the downstream products of HMG-CoA.

HMG-CoA is the rate limiting enzyme in the mevalonate pathway, which, through three branches, leads to the synthesis of cholesterol, dolichol (the precursor to dolichol pyrophosphate, which is the first thing added to proteins in post-translational glycosylation), and to ubiquinone, also known as Coenzyme Q (found in the membranes of endoplasmic reticulum, peroxisomes, lysosomes, vesicles and notably the inner membrane of the mitochondrion where it is an important part of the electron transport chain; it is also has important antioxidant activities).

However, it is likely that depletion of CoQ leads to a breakdown in the electron transport chain, leading in turn to a buildup in NADH, and a depletion in NAD⁺. Further, the reduced form of CoQ10, CoQ10H2, has an important cellular antioxidant function, which is to protect membranes and plasma lipoproteins against free radical-induced oxidation.

In some embodiments, the invention provides a method of reducing toxicity induced by a HMGCoA reductase inhibitor in a mammal, which method comprises administering to the mammal a therapeutically effective amount of a compound of the invention, wherein the mammal has been administered the HMGCoA reductase inhibitor in an amount that produces toxicity in the mammal in the absence of the administration of the compound of formula (I), and wherein the administration of the compound of claim 1 reduces the toxicity in the mammal. In some embodiments, the invention provides a method of reducing toxicity induced by a HMGCoA reductase inhibitor in a mammal, which method comprises administering to the mammal a therapeutically effective amount of a compound of the invention and then administering to the mammal the HMGCoA reductase inhibitor in an amount that produces toxicity in the mammal in the absence of the administration of the compound of formula (I), whereby toxicity that would have been induced by the HMGCoA reductase inhibitor is reduced in the mammal by the administration of the compound of the invention. In some embodiments, the invention provides a method of reducing toxicity induced by a HMGCoA reductase inhibitor in a mammal, which method comprises administering to the mammal a therapeutically effective amount of a compound of the invention, whereby toxicity induced by the HMGCoA inhibitor is reduced in the mammal, wherein the compound of the invention is administered to the mammal following manifestation of toxicity by the mammal.

In some embodiments, the invention provides a method of reducing toxicity induced by a genotoxic agent in a mammal, which method comprises administering to the mammal a therapeutically effective amount of a compound of the invention, wherein the mammal has been administered the genotoxic agent in an amount that produces toxicity in the mammal in the absence of the administration of the compound of the invention, and wherein the administration of the compound reduces the toxicity in the mammal. The compound of the invention can be administered to the mammal prior to administration of the genotoxic or other toxic agent to the mammal, simultaneously with administration of the genotoxic or other toxic agent to the mammal, or after administration of the genotoxic or other toxic agent to the mammal, for example, after symptoms of toxicity resulting from administration of the genotoxic or other toxic agent appear in the mammal.

In some embodiments, the invention relates to the use of a compound of the invention to prevent adverse effects and protect cells from toxicity. Toxicity may be an adverse effect of radiation or external chemicals on the cells of the body. Examples of toxins are pharmaceuticals, drugs of abuse, and radiation, such as UV or X-ray light. Both radiative and chemical toxins have the potential to damage biological molecules such as DNA. This damage typically occurs by chemical reaction of the exogenous agent or its metabolites with biological molecules, or indirectly through stimulated production of reactive oxygen species (e.g., superoxide, peroxides, hydroxyl radicals). Repair systems in the cell excise and repair damage caused by toxins.

Enzymes that use NAD⁺ play a part in the DNA repair process. Specifically, the poly(ADP-ribose) polymerases (PARPs), particularly PARP-1, are activated by DNA strand breaks and affect DNA repair. The PARPs consume NAD⁺ as an adenosine diphosphate ribose (ADPR) donor and synthesize poly(ADP-ribose) onto nuclear proteins such as histones and PARP itself. Although PARP activities facilitate DNA repair, overactivation of PARP can cause significant depletion of cellular NAD⁺, leading to cellular necrosis. The apparent sensitivity of NAD⁺ metabolism to genotoxicity has led to pharmacological investigations into the inhibition of PARP as a means to improve cell survival. Numerous reports have shown that PARP inhibition increases NAD+ concentrations in cells subject to genotoxicity, with a resulting decrease in cellular necrosis. Nevertheless, cell death from toxicity still occurs, presumably because cells are able to complete apoptotic pathways that are activated by genotoxicity. Thus, significant cell death is still a consequence of DNA/macromolecule damage, even with inhibition of PARP. This consequence suggests that improvement of NAD⁺ metabolism in genotoxicity can be partially effective in improving cell survival but that other players that modulate apoptotic sensitivity, such as sirtuins, may also play important roles in cell responses to genotoxins.

Physiological and biochemical mechanisms that determine the effects of chemical and radiation toxicity in tissues are complex, and evidence indicates that NAD⁺ metabolism is an important player in cell stress response pathways. For example, upregulation of NAD⁺ metabolism, via nicotinamide/nicotinic acid mononucleotide (NMNAT) overexpression, has been shown to protect against neuron axonal degeneration, and nicotinamide used pharmacologically has been recently shown to provide neuron protection in a model of fetal alcohol syndrome and fetal ischemia. Such protective effects could be attributable to upregulated NAD⁺ biosynthesis, which increases the available NAD⁺ pool subject to depletion during genotoxic stress. This depletion of NAD⁺ is mediated by PARP enzymes, which are activated by DNA damage and can deplete cellular NAD⁺, leading to necrotic death. Another mechanism of enhanced cell protection that could act in concert with upregulated NAD⁺ biosynthesis is the activation of cell protection transcriptional programs regulated by sirtuin enzymes.

Examples of cell and tissue protection linked to NAD⁺ and sirtuins include the finding that SIRT1 is required for neuroprotection associated with trauma and genotoxicity. SIRT1 can also decrease microglia-dependent toxicity of amyloid-beta through reduced NFKB signaling. SIRT1 and increased NAD⁺ concentrations provide neuroprotection in a model of Alzheimer's disease. Sirtuins are NAD⁺-dependent enzymes that have protein deacetylase and ADP-ribosyltransferase activities that upregulate stress response pathways. Evidence indicates that SIRT1 is upregulated by calorie restriction and in humans could provide cells with protection against apoptosis via downregulation of p53 and Ku70 functions. In addition, SIRT1 upregulates FOXO-dependent transcription of proteins involved in reactive oxygen species (ROS) detoxification, such as MnSOD. The sirtuin SIRT6 has been shown to participate in DNA repair pathways and to help maintain genome stability.

Pharmacological agents that target both NAD⁺ metabolism and sirtuins can provide tools to elucidate the involvement of these factors in modulating toxicity-induced tissue damage. Moreover, therapeutic options for treatment of acute and chronic tissue-degenerative conditions can emerge if sirtuins and NAD⁺ metabolism can be validated as providing enhanced tissue protection. Agents such as the plant polyphenols (e.g., resveratrol), the niacin vitamins, and the compound nicotinamide riboside can enhance cell survival outcomes by increasing NAD⁺ biosynthesis, reducing NAD⁺ depletion, and/or activating sirtuin enzymes.

In another embodiment, the invention provides a process for the preparation of a compound of formula (I):

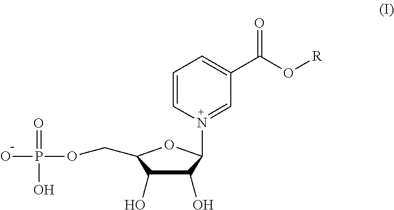

wherein R is methyl or ethyl, or a salt thereof, wherein the process comprises the steps of:

(i) reacting a nicotinate ester (IV):

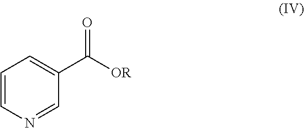

with 1,2,3,4-tetra-O-acetyl-D-ribofuranose to provide a compound of formula (V):

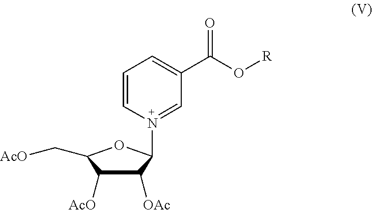

(ii) reacting the compound of formula (V) with a base to form the compound of formula (III):

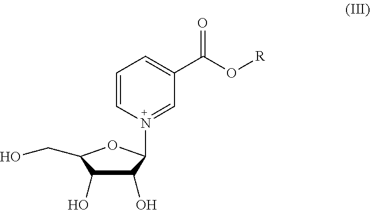

and
(iii) reacting the compound of formula (III) with a mixture of POCl₃ and PO(OR⁵)_{3,} wherein R⁵ is C₁-C₆ alkyl, followed by treatment with water to form the compound of formula (I).

In certain of these embodiments, R⁵ is ethyl.

In an embodiment, the compound of formula (I) can be synthesized as shown in Scheme 1.

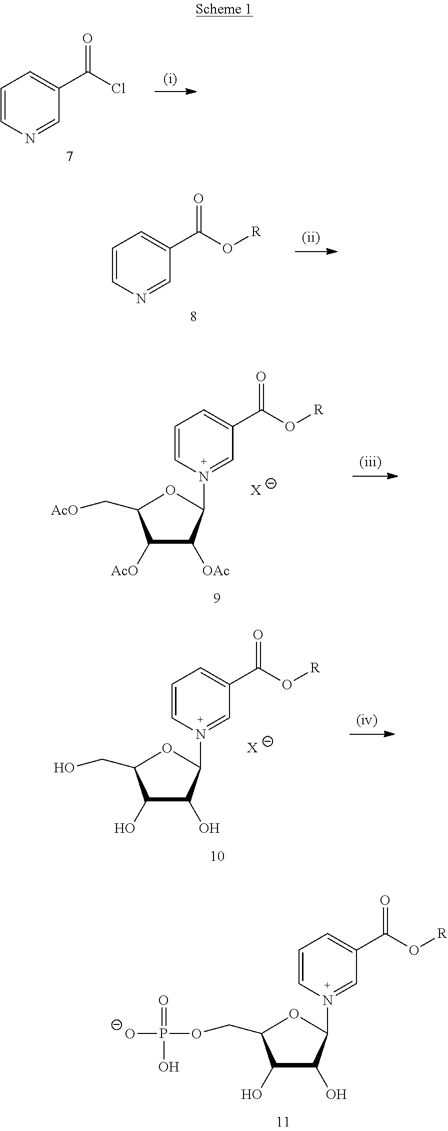

Reagents and conditions: (a): alcohols, triethylamine, 4-dimethylaminopyridine, DCM, -78° C. to reflux; (b): β-D-ribofuranose 1,2,3,5-tetraacetate, trimethylsilyl trifluoromethanesulfonate, DCM, reflux; (c): NaOEt, EtOH, −20° C.; (d): POCl₃, triethyl phosphate

Nicotinate ester 8 can be prepared using any suitable method. For example, nicotinoyl chloride 7 can be reacted with methanol or ethanol in the presence of a base such as trimethylamine and a basic catalyst such as 4-dimethylaminopyridine in a suitable solvent such as dichloromethane (DCM). Protected triacetyl nicotinate riboside 9 can be prepared by reacting nicotinate ester 8 with an acetylated riboside such as 1,2,3,5-tetra-O-acetyl-D-ribofuranose in the presence of a catalyst such as trimethylsilyl trifluoromethanesulfonate in a suitable solvent such as DCM to provide 9. Triacetyl nicotinate riboside 9 can be deprotected by reaction with a base sodium ethoxide in a solvent such as ethanol (EtOH) to provide deprotected nicotinate riboside 10.

Compound 10 can be phosphorylated using any suitable conditions. Preferably, compound 10 can be phosphorylated in a mixture of phosphorus oxychloride and PO(OR⁵)₃, wherein R⁵ is C₁-C₆ alkyl. Preferably, compound 10 is phosphorylated in a mixture of phosphorus oxychloride (POCl₃) and triethylphosphate to provide compound 11 (i.e., a compound of formula (I)). The phosphorylation can be conducted at any suitable temperature. For example, the phosphorylation can be conducted at about −20° C. to about 50° C. and is preferably conducted at 0° C.

Compound 11 can be isolated using any suitable isolation technique. For example, compound 11 can be isolated by precipitation of compound 11 from an aqueous mixture or solution by the addition of a suitable solvent such as ethyl acetate, tetrahydrofuran, acetonitrile, and the like, followed by filtration to obtain compound 11 as a solid. Other isolation techniques, such as high performance liquid chromatography (HPLC) can also be used to isolate compound 11.

In an embodiment, O-methyl nicotinamide riboside mononucleotide 12 can be prepared from the corresponding O-ethyl compound 11 (R=Et) by ester exchange, for example, using NaOMe in MeOH at −20° C., as shown in Scheme 2.

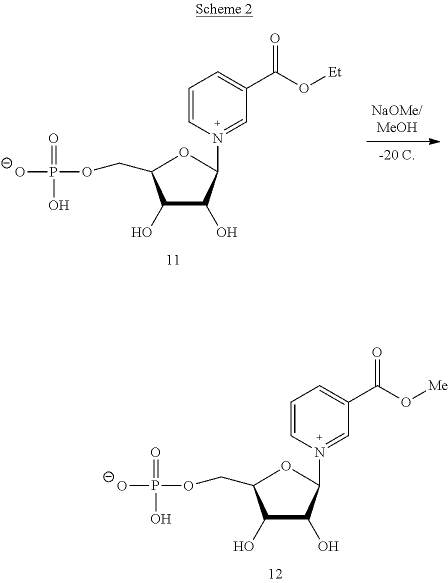

### Preferred Embodiments

The invention includes the following embodiments:

1. A compound of formula (I):

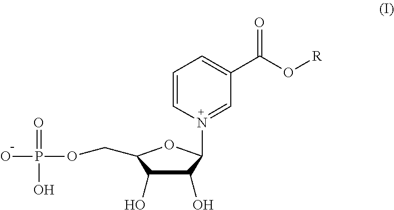

wherein R is methyl or ethyl, or a salt thereof.

2. The compound or salt of embodiment 1, wherein the compound is:

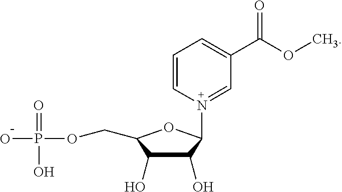

3. The compound or salt of embodiment 1, wherein the compound is:

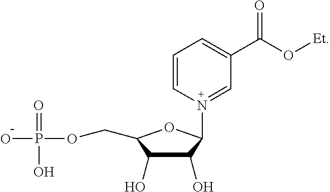

4. A pharmaceutical composition comprising the compound or salt of any one of embodiments 1-3 and a pharmaceutically acceptable carrier.

5. A nutraceutical composition comprising a compound or salt of any one of embodiments 1-3.

6. A method for increasing cell NAD⁺ production comprising administering to a cell a compound of formula (I):

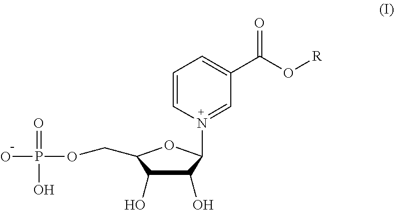

wherein R is methyl or ethyl, or a salt thereof.

7. The method of embodiment 6, wherein the compound is:

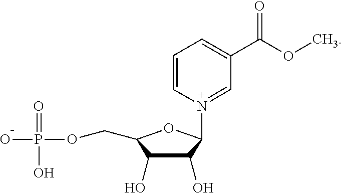

8. The method of embodiment 6, wherein the compound is:

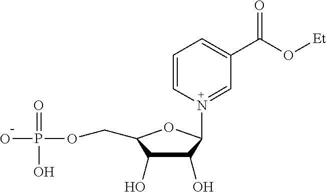

9. The method of any one of embodiments 6-8, wherein the cell is in a mammal having a lipid disorder, a metabolic dysfunction, a cardiovascular disease, CNS or PNS trauma, a neurodegenerative disease or condition, or hearing loss, or is in a mammal that has been exposed to a toxic agent.

10. The method of any one of embodiments 6-8, wherein the cell is in a mammal at risk for hearing loss.

11. The method of any one of embodiments 6-8, wherein the cell is in a mammal, wherein the compound is administered in an amount effective for promoting the function of the metabolic system, promoting muscle function or recovery, promoting the function of the auditory system, or promoting cognitive function.

12. A method of improving mitochondrial densities in a cell, wherein the method comprises administering to the cell a compound of formula (I):

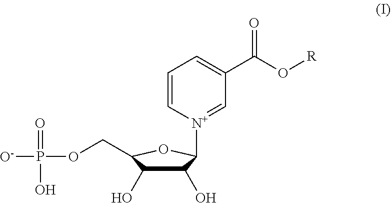

wherein R is methyl or ethyl, or a salt thereof.

13. The method of embodiment 12, wherein the compound is:

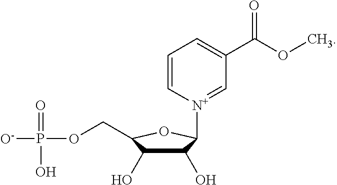

14. The method of embodiment 12, wherein the compound is:

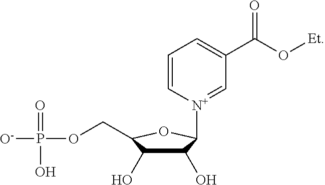

15. The method of any one of embodiments 12-14, wherein the cell is in a mammal having a lipid disorder, a metabolic dysfunction, a cardiovascular disease, CNS or PNS trauma, a neurodegenerative disease or condition, hearing loss, or is in a mammal that has been exposed to a toxic agent.

16. The method of any one of embodiments 12-14, wherein the cell is in a mammal at risk for hearing loss.

17. The method of any one of embodiments 12-14, wherein the cell is in a mammal, wherein the compound is administered in an amount effective for promoting the function of the metabolic system, promoting muscle function or recovery, promoting the function of the auditory system, or promoting cognitive function.

18. A process for the preparation of a compound of formula (I):

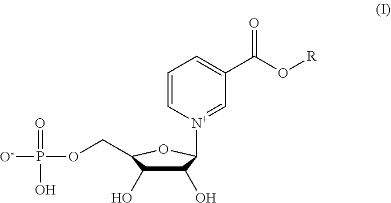

wherein R is methyl or ethyl, or a salt thereof, wherein the process comprises the steps of:

(i) reacting a nicotinate ester (IV):

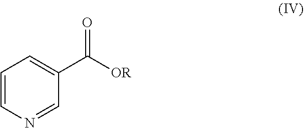

with 1,2,3,4-tetra-O-acetyl-D-ribofuranose to provide a compound of formula (V):

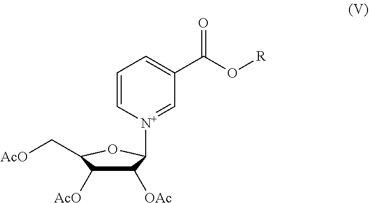

(ii) reacting the compound of formula (V) with a base to form the compound of formula (III):

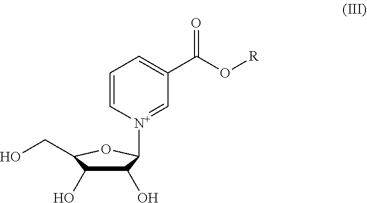

and

(iii) reacting the compound of formula (III) with a mixture of POCl₃ and PO(OR⁵)_{3,} wherein R⁵ is C₁-C₆ alkyl, followed by treatment with water to form the compound of formula (I).

19. A process for the preparation of a compound of formula (I):

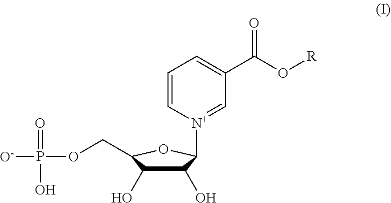

wherein R is methyl, or a salt thereof, wherein the process comprises the steps of:

(i) reacting a nicotinate ester (IV):

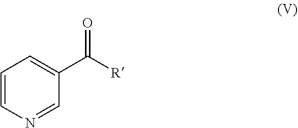

with 1,2,3,4-tetra-O-acetyl-D-ribofuranose to provide a compound of formula (VI):

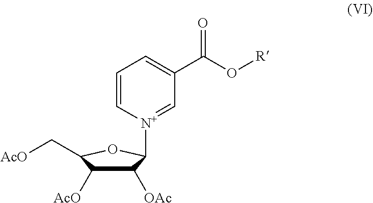

(ii) reacting the compound of formula (VI) with a base to form the compound of formula (VII):

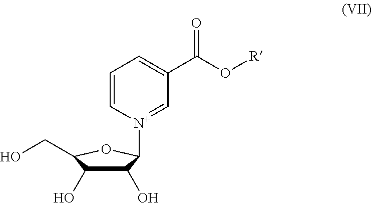

(iii) reacting the compound of formula (VII) with a mixture of POCl₃ and PO(OR⁵)_{3,} wherein R⁵ is C₁-C₆ alkyl, followed by treatment with water to form the compound of formula (VIII):

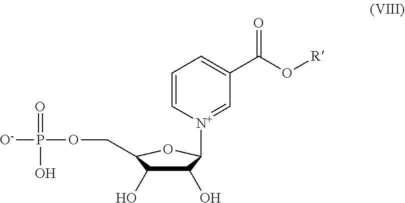

and

(iv) reacting the compound of formula (VIII) with sodium methoxide to form the compound of formula (I).

### EXAMPLES

The following examples further illustrate the invention but, of course, should not be construed as in any way limiting its scope.

### Example 1

This example demonstrates a general synthesis of alkyl (β-nicotinic ribosides.

Methyl (β-nicotinic riboside and ethyl β-nicotinic riboside were synthesized as described in U.S. Pat. No. 8,106,184, the disclosure of which is incorporated herein by reference.

General procedure for the synthesis of nicotinate riboside mononucleotides. To a solution of nicotinate riboside in triethyl phosphate was added 2.5 eq. POCl₃ at 0° C. The reaction mixture was stirred at 0° C. for 16 h and then neutralized with cold saturated NaHCO₃ solution (pH=7). The reaction mixture was concentrated under reduced pressure to minimum volume, and ethyl acetate was added thereto. Filtration afforded pure nicotinate riboside mononucleotide as a solid.

### Example 2

This example demonstrates a synthesis of ((2R,3R,4S,5R)-3,4-dihydroxy-5-(3-(ethoxycarbonyl)pyridin-1-ium-1-yl)tetrahydrofuran-2-yl)methyl hydrogen phosphate.

1-((2R,3S,4R,5R)-3,4-dihydroxy-5-(hydroxymethyl)tetrahydrofuran-2-yl)-3-(ethoxycarbonyl)pyridin-1-ium trifluoromethanesulfonate (20 mg, 0.085 mm) and triethyl phosphate (1 ml) were placed in a flame-dried round-bottom flask. The mixture was cooled to 0° C., and POCl₃ (65 mg, 0.4 mmol) was added dropwise to the mixture. The reaction mixture was stirred at the same temperature for 16 h. After completion, the reaction mixture was neutralized with cold saturated NaHCO₃ solution (pH=7). The resulting solution was directly concentrated under reduced pressure to minimum volume, and 2 ml of ethyl acetate was added thereto. Filtration afforded pure compound as a white solid. ¹H NMR (500 MHz, D₂O): 67 .

### Example 3

This example demonstrates the effect of nicotinic acid mononucleotide on intracellular NAD⁺ levels in HEK293 and Neuro2a cells.

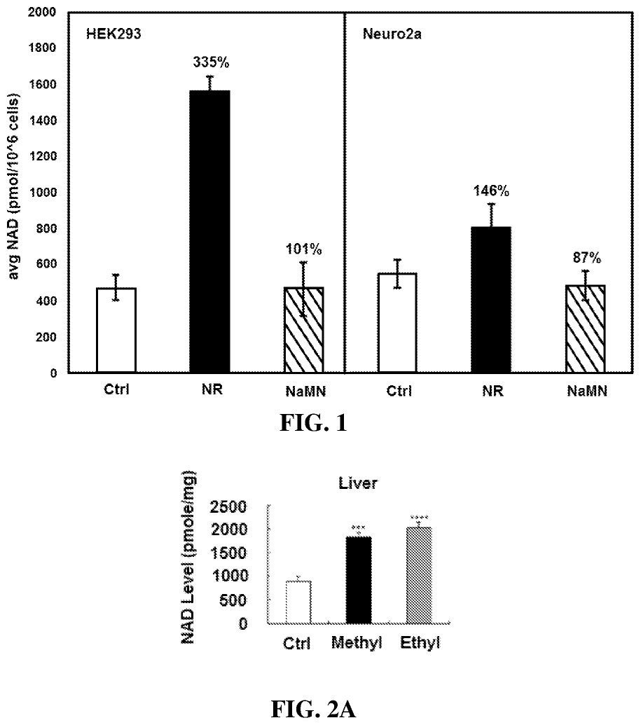

The ability of nicotinic acid mononucleotide, NaMN, to serve as an NAD⁺ enhancing agent in mammalian cell lines, Neuro2a and HEK293 cells was examined after an 8 h incubation. NaMN was added to cell growth media at a concentration of 1 mM. All cell treatments were done in duplicate. Nicotinamide riboside (NR) at 1 mM concentration was performed as a positive control. Untreated cells served as negative controls. Cells were treated for the allotted time then harvested by trysin detachment and pelleting. Cells were counted by haemocytometer and then lysed by treatment with 100 μL perchloric acid (7%). Lysates were then neutralized by treatment with NaOH and K₂PO₄ solutions. NAD⁺ concentrations were determined by a diaphorase-based assay. NAD⁺ standards were also run to establish a standard curve. The results are graphically depicted in FIG. 1.

As is apparent from the results shown in FIG. 1, only NR treatment results in increased NAD⁺ contents in cells. No intracellular increase in NAD⁺ is observed with NaMN treatment. These results indicate that the acid compound NaMN does not appear to act as an NAD⁺ precursor or NAD⁺ enhancer when applied externally to mammalian cells.

All references, including publications, patent applications, and patents, cited herein are hereby incorporated by reference to the same extent as if each reference were individually and specifically indicated to be incorporated by reference and were set forth in its entirety herein.

### Example 4

This example demonstrates the effect of compounds of the invention on NAD⁺ concentration in HEK-293 cells (human embryonic kidney), in accordance with an embodiment of the invention.

HEK-293 cells were treated with 0 μM, 250 μM, and 500 μM of ((2R,3R,4S,5R)-3,4-dihydroxy-5-(3-(ethoxycarbonyl)pyridin-1-ium-1-yl)tetrahydrofuran-2-yl)methyl hydrogen phosphate (EtNaMN) at an initial density of 500,000 cells per well. After 24 h treatment, the NAD⁺ concentration was determined using a known NAD⁺ cycling assay (Jacobson, E. L. et al., Arch. Biochem. Biophys., 175: 627-634 (1976)). The cells were washed with PBS, counted by haemocytometry and pelleted. The cells were then treated with 7% perchloric acid, and then neutralized with 1 M NaOH and 500 mM potassium phosphate pH 8.5. NAD⁺ contents were then measured on a plate reader using diaphorase and lactate dehydrogenase using Resazurin as a dye that is reduced to rezarufin (emission 560 nm). NAD⁺ levels were quantitated using a standard curve using known NAD⁺ concentrations. NAD⁺ concentrations are determined to nmol NAD⁺/10⁶ cells. The NAD⁺ concentrations and levels relative to the control treatment are set forth in Table 1.

TABLE 1
NAD⁺ concentrations in HEK293
cells after treatment with EtNaMN

| Concentration | NAD⁺ | NAD⁺Concentration |
|---|---|---|
| of EtNaMN | Concentration | relative to control |
| 0 μM (control) | 170 μM ± 30 μM | 1.00 ± 0.17 |
| 250 μM | 220 μM ± 42 μM | 1.29 ± 0.24 |
| 500 μM | 270 μM ± 35 μM | 1.59 ± 0.21 |

As is apparent from the results set forth in Table 1, treatment of HEK293 cells with 250 μM and 500 μM of EtNaMN resulted in increases in intracellular NAD⁺ levels of 1.29 and 1.59, respectively, relative to control.

### Example 5

This example demonstrates a synthesis of ((2R,3R,4S,5R)-3,4-dihydroxy-5-(3-(ethoxycarbonyl)pyridin-1-ium-1-yl)tetrahydrofuran-2-yl)methyl hydrogen phosphate (O-ethyl nicotinate riboside; O-ethyl NAR; EtNaMN).

(a) Synthesis of Ethyl Nicotinate-Riboside

O-EthylNAR was obtained by an altered synthesis from a previously described method (Yang et al., J. Med. Chem. 50: 6458-6461 (2007)). 2′, 3′, 5′-Triacetyl ethyl nicotinate riboside (MW=559, TFA salt, 2 g, 3.5 mmol) was dissolved into 20 mL of 300 mM NaOEt/EtOH on ice. After mixing, the reaction was stored at −20° C. for 12 hours. The reaction was quenched with addition of 2% ethanolic HCl to acidify. Organic solvent was removed by vacuum and redissolved in ethanol. Insoluble NaCl was removed by filtration. The residue was dissolved in water, and extracted with cyclohexane to remove organic impurities. Lyophilization produced a pure substance for further modification.

(b) Synthesis of Ethyl Nicotinate-Riboside-Mono-phosphate

1.25 grams of O-EthylNAR triflate salt (MW=433, 2.9 mmols) was dissolved in 20 mL of anhydrous triethylphosphate, in a flame dried round bottom flask containing a stirbar under an Ar atmosphere. The reaction mixture was cooled by an icebath to 0° C. The reaction was stirred and 2 equivalents of phosphorus oxychloride (POCl_{3,} MW=153, 887 mg, 5.8 mmol) was added dropwise into the reaction mixture via syringe. The reaction was kept cool for 48 hours until reaction was 90% complete by HPLC. Reaction was subsequently quenched by addition of ice, and the reaction mixture was neutralized by addition of 1 M NaOH. The reaction was then extracted with 40 mL water and 100 mL ethylacetate with 2 additional 100 mL ethylacetate extractions to remove the triethylphosphate solvent. The water layer containing product was dried under reduced pressure. The residue was added to a 100 mL C-18 column equilibrated with water. Elution of 150 mL of water was followed by elution with 10% methanol/water which eluted product. Yield was 70% after lyophilization. ¹H NMR (500 MHz, D₂O) δ 9.49 (s, 1H), 9.29 (d, J=6.3 Hz, 1H), 9.05 (d, J=8.0, 1H), 8.25 (dd, J=8.1, 6.2 Hz, 1H), 6.16 (d, J=4.7 Hz, 1H), 4.51−4.45 (m, 4H), 4.37 (dd, J=4.9, 3.4 Hz, 1H), 4.25 (dd, J=12.3, 2.7 Hz, 1H), 4.11 (dd, J=12.3, 2.3 Hz, 1H), 1.34 (t, 3H). LCMS=m/z=364.1 (Molecular ion +1).

### Example 6

This example demonstrates a synthesis of ((2R,3R,4S,5R)-3,4-dihydroxy-5-(3-(methoxycarbonyl)pyridin-1-ium-1-yl)tetrahydrofuran-2-yl)methyl hydrogen phosphate (O-methyl nicotinate riboside; O-methyl NAR; MeNaMN).

To a flame dried 50 mL round bottom flask was added 500 mg of O-Ethyl-Nicotinate Riboside-5-phosphate (MW=363, 1.38 mmol) and the flask was sealed by a rubber stopper. With N₂ ventilation, 10 mL of anhydrous methanol was added by syringe. The reaction was cooled to −20° C. In a separate flask 50 mg of sodium metal (MW=23, 2.17 mM) was added to 10 mL of methanol at 0° C. The contents of the second flask were added to the first after a clear solution and cessation of bubbling was obtained. The reaction was permitted to react 14 hr at −20° C. The reaction was quenched by addition of 10 mL 10% acetic acid dissolved in methanol. The reaction mixture was then dried by vacuum to provide a yellowish residue. The residue was redissolved in water, and then purified by elution on a C-18 gravity column having a volume of 100 mL. Initial elutions were of water, followed by 10% methanol/water. The 10% fractions eluted the desired product. Fractions were combined and lyophilized to produce a pure uncolored substance in excellent yield (80% versus O-EthylNaMN). HPLC, NMR and LCMS established purity and identity. NMR Spectrum shown as FIG. 1. ¹ H NMR (500 MHz, D₂O) δ 9.49 (s, 1H), 9.30 (d, J=6.3 Hz, 1H), 9.06(d, J=8.0 Hz 1H), 8.27 (dd, J=8.1, 6.2 Hz, 1H), 6.16 (d, J=4.7 Hz, 1H), 4.71−4.45 (m, 2H), 4.37 (dd, J=4.9, 3.4 Hz, 1H), 4.17 (dd, J=12.3, 2.7 Hz, 1H), 4.12 (dd, J=12.3, 2.3 Hz, 1H), 4.08 (s, 3H). δ 163.39, 147.31, 143.18, 141.87, 132.13, 128.72, 102.83, 87.48, 77.77, 70.95, 64.15, 53.95. LCMS=m/z=350.2 (Molecular ion+1).

### Example 7

This example demonstrates the effects of O-methyl nicotinate riboside and O-ethyl nicotinate riboside on tissue NAD concentrations after i.p. administration of the compounds to mice.

Sterile solutions of O-methyl-NaMN and O-ethylNaMN were prepared by combination with phosphate buffered saline for injection into mice. Mice were injected by intraperitoneal injection at a single dosage of 500 mg/kg. All mice were weighed to achieve correct injection volume. Each group was composed of 5 mice, and mice were sacrificed at 4 hours. Control mice received only PBS. At sacrifice, mice were first injected with an anesthetic dose of ketamine to obtain blood by cardiac puncture. Then mice were euthanized and tissues harvested and frozen immediately in liquid nitrogen.

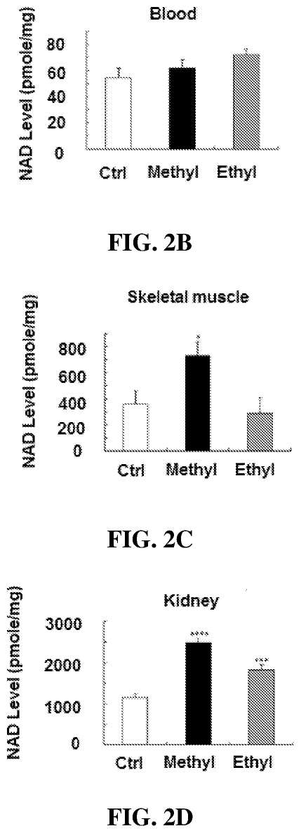

To determine tissue NAD concentrations, tissues were weighed and pulverized in liquid nitrogen. Perchloric acid (7%) was then added to pulverized tissue samples and neutralized by NaOH and 250 mM potassium phosphate buffer pH 8.5. Samples were pelleted to remove insoluble material. NAD concentrations were determined by cycling assay as published (Li et al., Methods in molecular biology 1241, 39-48, doi:10.1007/978-1-4939-1875-1_4 (2015)). NAD concentrations were determined in pmol NAD/mg tissue and are displayed graphically in FIGS. 2A-2D. Significant increases in tissue NAD was observed for the ethyl derivative in kidney and liver. These increases were approximately twofold in kidney and greater than twofold in liver. On the other hand, increases in blood were not apparent after 4 hours. Moreover, the ethyl compound had no apparent effect on skeletal muscle NAD levels after 4 hour exposure. The methyl derivative was similar in effect for liver to ethyl, but not as great. On the other hand, the methyl compound had the unique capability to increase skeletal muscle NAD concentrations, an effect absent from the ethyl compound. This property is desirable, as effects expected from NAD enhancement in muscle include mitochondrial biogenesis and improved insulin sensitivity, as noted for other agents that increase NAD concentrations in this tissue³. In fact, the extent of NAD raising exceeds those observed for other agents. The effect of methyl in kidney is also impressive, approaching a 2.5 fold increase, and portending potential action for protection in diseases of kidney and in kidney failure, where recent work suggests potential for NAD enhancers to provide a therapeutic benefit. Neither ethyl nor methyl derivatives appeared active as NAD enhancers in blood, at least within the 4 hour period of exposure.

### Example 8

This example demonstrates the effects of O-methyl nicotinate riboside and O-ethyl nicotinate riboside on cellular NAD concentrations at 14 hours in Neu2A, HEK293, HEPG2, and C2C12 cells.

Neu2a, HEK293, HEPG2 and C2C12 cells were cultured and the dosage of the corresponding compound was increased by amounts of 0, 100 μM, 400 μM and 800 μM for each cell line. Each experiment was performed in triplicate. Cells were incubated for a period of 14 hr. At termination of incubation, media was removed and cells were treated with trypsin to gently detach and cells counted. Cell pellets were obtained by centrifugation, and then 100 mL of 7% perchloric acid was added to extract NAD. Extracts obtained were then neutralized by addition of NaOH and 250 mM potassium phosphate buffer pH 8.5. NAD was determined by a cycling assay that measures NAD concentrations by comparison to a standard curve (Li et al., 2015). The results of these incubations are set forth in Table 2.

TABLE 2
Effects of O-methyl nicotinate riboside (O-MeNaMN) and O-ethyl nicotinate riboside
(O-EtNaMN) on cellular NAD in Neu2A, HEK293, HEPG2, and C2C12 cells

| Compound | Cell line | 0 μM | 100 μM | 400 μM | 800 μM |
|---|---|---|---|---|---|
| O-MeNaMN | C2C12 | 1522 ± 250 | 2864 ± 565 | 2596 ± 572 | 2574 ± 355 |
| O-MeNaMN | HEK293 | 824 ± 74.5 | 1178 ± 65 | 947 ± 101 | 1440 ± 204 |
| O-MeNaMN | HEPG2 | 1757 ± 261 | 2227 ± 280 | 1663 ± 61 | 2383 ± 155 |
| O-MeNaMN | Neu2a | 961 ± 58.6 | 1204 ± 158 | 1523 ± 145 | 2125 ± 187 |
| O-EtNaMN | C2C12 | 1373 ± 135 | 1886 ± 183 | 1766 ± 69.5 | 2320 ± 208 |
| O-EtNaMN | HEK293 | 781 ± 54.5 | 1128 ± 95 | 1144 ± 132 | 1133 ± 168 |
| O-EtNaMN | HEPG2 | 1656 ± 250 | 2222 ± 99 | 2331 ± 429 | 2287 ± 181 |
| O-EtNaMN | Neu2a | 1170 ± 113 | 1624 ± 24 | 1727 ± 40.8 | 1744 ± 151 |

As is apparent from the results set forth in Table 2, statistically significant increases were observed in all cell lines by at least one concentration of compound in all instances. The weakest of cell lines for responsiveness was HEPG2 although it increased NAD concentrations versus controls. Neu2a cells responded well to both O-MethylNaMN as well as O-EthylNaMN and increases showed dose dependency, with increases rising almost 40% at 100 μM for the Ethyl derivative. Corresponding increases in concentration of ethyl and methyl compound (800 μM) caused cellular NAD concentrations to increase more than two fold for the methyl compound, and to 50% for the ethyl compound. At all methyl compound concentrations tested cellular NAD concentrations increased by at least 70%. The cellular enhancement achieved by the ethyl compound showed dose dependence and far less effect, although it could still raise NAD concentrations by 68% at the 800 μM dose. Collectively these data indicate that both methyl and ethyl ester derivatives of NaMN exhibit pharmacologic activity as NAD enhancers in cell culture. Some structure activity effect is obtained by modification of the ester, as seen for C2C12 cells, a murine muscle-origin cell line. The highest increases in cellular NAD concentration in all cell lines was generally observed for the methyl derivative, especially at lowest and highest dosages.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 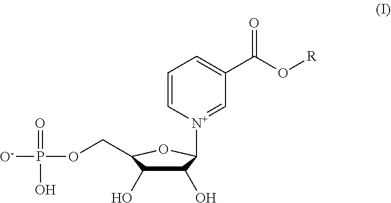
wherein R is methyl or ethyl, or a salt thereof.

2. The compound or salt of claim 1, wherein the compound is:

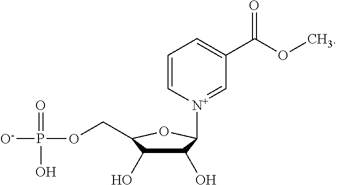

3. The compound or salt of claim 1, wherein the compound is:

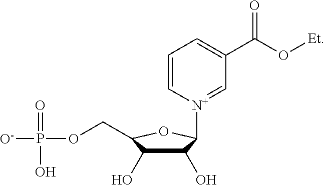

4. A pharmaceutical composition comprising the compound or salt of claim 1 and a pharmaceutically acceptable carrier.

5. A nutraceutical composition comprising a compound or salt of claim 1.

6. A method for increasing cell NAD⁺ production comprising administering to a cell a compound of formula (I): 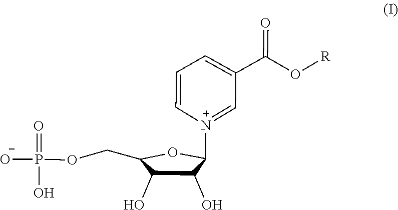
wherein R is methyl or ethyl, or a salt thereof.

7. The method of claim 6, wherein the compound is:

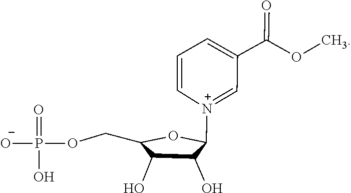

8. The method of claim 6, wherein the compound is:

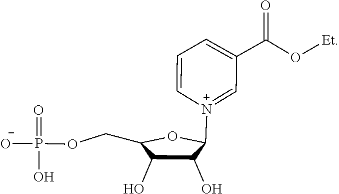

9. The method of claim 6, wherein the cell is in a mammal having a lipid disorder, a metabolic dysfunction, a cardiovascular disease, CNS or PNS trauma, a neurodegenerative disease or condition, or hearing loss, or is in a mammal that has been exposed to a toxic agent.

10. The method of claim 6, wherein the cell is in a mammal at risk for hearing loss.

11. The method of claim 6, wherein the cell is in a mammal, wherein the compound is administered in an amount effective for promoting the function of the metabolic system, promoting muscle function or recovery, promoting the function of the auditory system, or promoting cognitive function.

12. A method of improving mitochondrial densities in a cell, wherein the method comprises administering to the cell a compound of formula (I): 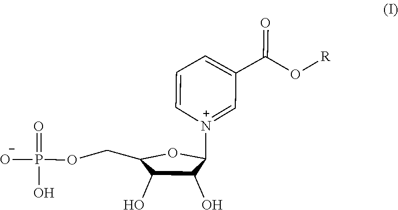
wherein R is methyl or ethyl, or a salt thereof.

13. The method of claim 12, wherein the compound is:

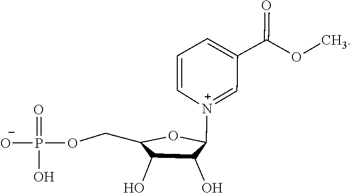

14. The method of claim 12, wherein the compound is:

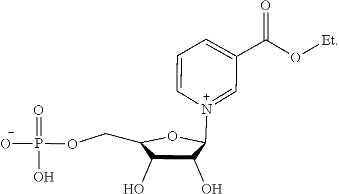

15. The method of claim 12, wherein the cell is in a mammal having a lipid disorder, a metabolic dysfunction, a cardiovascular disease, CNS or PNS trauma, a neurodegenerative disease or condition, hearing loss, or is in a mammal that has been exposed to a toxic agent.

16. The method of claim 12, wherein the cell is in a mammal at risk for hearing loss.

17. The method of claim 12, wherein the cell is in a mammal, wherein the compound is administered in an amount effective for promoting the function of the metabolic system, promoting muscle function or recovery, promoting the function of the auditory system, or promoting cognitive function.

18. A process for the preparation of a compound of formula (I): 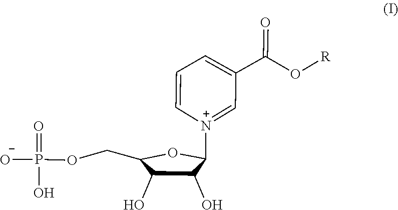
wherein R is methyl or ethyl, or a salt thereof, wherein the process comprises the steps of:
(i) reacting a nicotinate ester (IV): 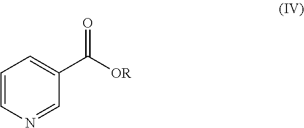
with 1,2,3,4-tetra-O-acetyl-D-ribofuranose to provide a compound of formula (V): 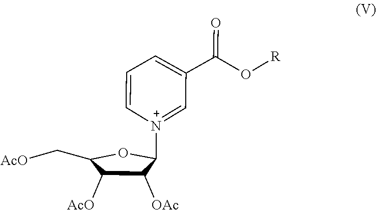
(ii) reacting the compound of formula (V) with a base to form the compound of formula (III): 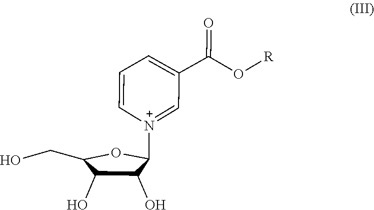
and
(iii) reacting the compound of formula (III) with a mixture of POCl₃ and PO(OR⁵)_{3,} wherein R⁵ is C₁-C₆ alkyl, followed by treatment with water to form the compound of formula (I).

19. A process for the preparation of a compound of formula (I): 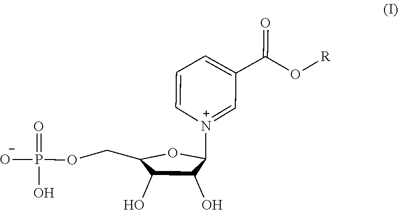
wherein R is methyl, or a salt thereof, wherein the process comprises the steps of:
(i) reacting a nicotinate ester (IV): 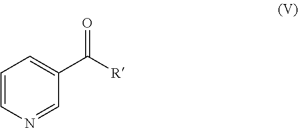
with 1,2,3,4-tetra-O-acetyl-D-ribofuranose to provide a compound of formula (VI): 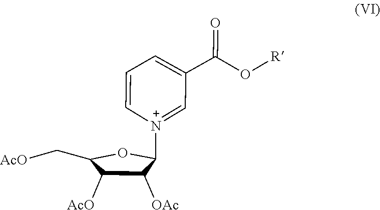
(ii) reacting the compound of formula (VI) with a base to form the compound of formula (VII): 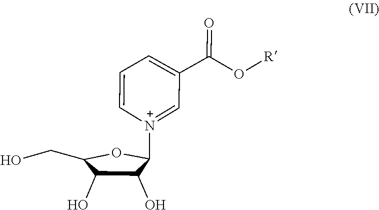
(iii) reacting the compound of formula (VII) with a mixture of POCl₃ and PO(OR⁵)_{3,} wherein R⁵ is C₁-C₆ alkyl, followed by treatment with water to form the compound of formula (VIII): 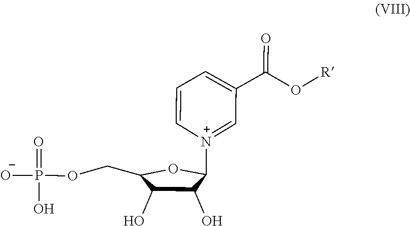
and
(iv) reacting the compound of formula (VIII) with sodium methoxide to form the compound of formula (I).

20. A pharmaceutical composition comprising the compound or salt of claim 2 and a pharmaceutically acceptable carrier.
